(19) United States
(12) Patent Application Publication
(10) Pub. No.: US 2016/0106308 A1
(43) Pub. Date: Apr. 21, 2016

(54) AIRWAY TUBE AND VISUALISATION DEVICE ASSEMBLY

(71) Applicant: SMITHS MEDICAL INTERNATIONAL LIMITED, Ashford, Kent (GB)

(72) Inventor: Stephen James Field, Kent (GB)

(73) Assignee: SMITHS MEDICAL INTERNATIONAL LIMITED, Kent TN25 4BF (GB)

(21) Appl. No.: 14/892,599

(22) PCT Filed: May 9, 2014
(86) PCT No.: PCT/GB2014/000180

§ 371 Date: Nov. 20, 2015

(30) Foreign Application Priority Data
Jun. 7, 2013 (GB) 1310158.9

(51) Int. Cl. A61B 1/267; A61B 1/05; A61B 1/00; A61M 16/04

(52) U.S. Cl. CPC A61B 1/267; A61M 16/0488; A61B 1/05; A61B 1/00114; A61B 1/00124; A61B 1/00117; A61B 1/00126; A61B 1/00165; A61B 1/0014

(57) ABSTRACT

A video introducer (2) is inserted in an endotracheal tube (1) and is retained by engagement of a connector (28) at the rear end of the introducer with a connector (16) inserted in the rear end (13) of the tube. The introducer (2) includes a shaft (21) integral with the connector (28) and having a channel (22) along its length. A cable (34) connected to a video camera (33) at the patient end of the shaft extends along the channel (22) to an electrical connector (35). The forward end (25) of the shaft (21) projects from the forward end (10) of the tube to provide a leading extension for inserting the tube in the trachea.

This invention relates to assemblies of the kind including an airway tube and a first connector fitted onto the rear end of the tube.

The invention is more particularly concerned with assemblies including a viewing device or visualisation means for use in placing apparatus within a patient such as inserting an endotracheal tube into the trachea. Traditional introducers or bougies used to insert a tube take the form of a simple rod that can be bent to an approximate desired shape and can flex to accommodate the shape of the anatomy during insertion. The introducer may be made with an angled, Coude tip to facilitate introduction. The introducer can be inserted more easily than the tube itself because it has a smaller diameter and can be bent and can flex to the ideal shape for insertion. These bougies may be used with or without the aid of a laryngoscope. When the bougie has been correctly inserted, a tube can be slid along its outside to the correct location, after which the bougie is pulled out of the tube, which is left in position. Bougies are available from Smiths Medical. GB2312378 describes an introducer or bougie moulded of an aliphatic polyurethane material and also describes an earlier bougie made from a braided polyester filament repeatedly coated in layers of resin. The characteristic flexural and recovery properties of these bougies are highly valued by clinicians. Introducers can alternatively be inserted within the tube before insertion in the patient and the assembly of the tube and introducer inserted into the patient together.

More recently it has been proposed to use fibre optics or a video camera with an introducer to provide the clinician with a view of the trachea as the introducer is inserted. WO2007/089491 describes an arrangement with an introducer and a separate camera assembly clipped onto the outside of the introducer, which is removed before an endotracheal tube can be slid along the introducer. WO2010/136748 describes an introducer arrangement with a camera at one end of an introducer and connected via a cable to a display screen at the opposite end.

It is an object of the present invention to provide an alternative assembly of an airway tube and visualisation means.

According to one aspect of the present invention there is provided an assembly of the above-specified kind, characterised in that the assembly includes a visualisation device including an imaging device at one end, a shaft extending along the visualisation device, a second connector towards the rear end of the shaft and mateable with the first connector, and a cable extending along the shaft from the imaging device to a signal connector located to the rear of the second connector, the second connector being removably fitted with the first connector on the tube and such that the imaging device is located towards the forward, patient end of the tube to view the field of view forwardly of the patient end of the tube.

The imaging device preferably includes a video camera, the cable being an electrical cable and the signal connector being an electrical connector. Alternatively, the imaging device may be provided by one end of an optical fibre cable, the signal connector being an optical connector. The shaft preferably has a channel opening along its length, at least a major part of the length of the cable being fitted in the channel. The channel may have a plurality of clip formations along its length to retain the cable in the channel. The visualisation device preferably projects beyond the forward end of the tube such that the imaging device is located forwardly of the forward end of the tube. A length of the visualisation device projecting beyond the forward end of the tube may provide a leading extension for use during insertion of the assembly. The width of the shaft may be less than the bore of the tube apart from a plurality of enlarged bosses that help centralise the shaft in the bore of the tube. The second connector preferably includes an opening that allows air to flow through the tube during use. The first connector preferably has a male fitting at one end that is fitted in the machine end of the tube and a male fitting at its opposite end that is fitted in the second connector. The second connector preferably has an opening therethrough, the signal connector being threaded through the opening. The second connector may have an external surface that provides a handle by which the assembly can be gripped and manipulated. The shaft and second connector may be integrally formed with one another as a single piece. Alternatively, the shaft may extend rearwardly beyond the second connector and be slidable relative to the second connector.

According to another aspect of the present invention there is provided a visualisation device for use in an assembly according to the above one aspect of the present invention.

An assembly of a tracheal tube and video introducer will now be described, by way of example, with reference to the accompanying drawings, in which:

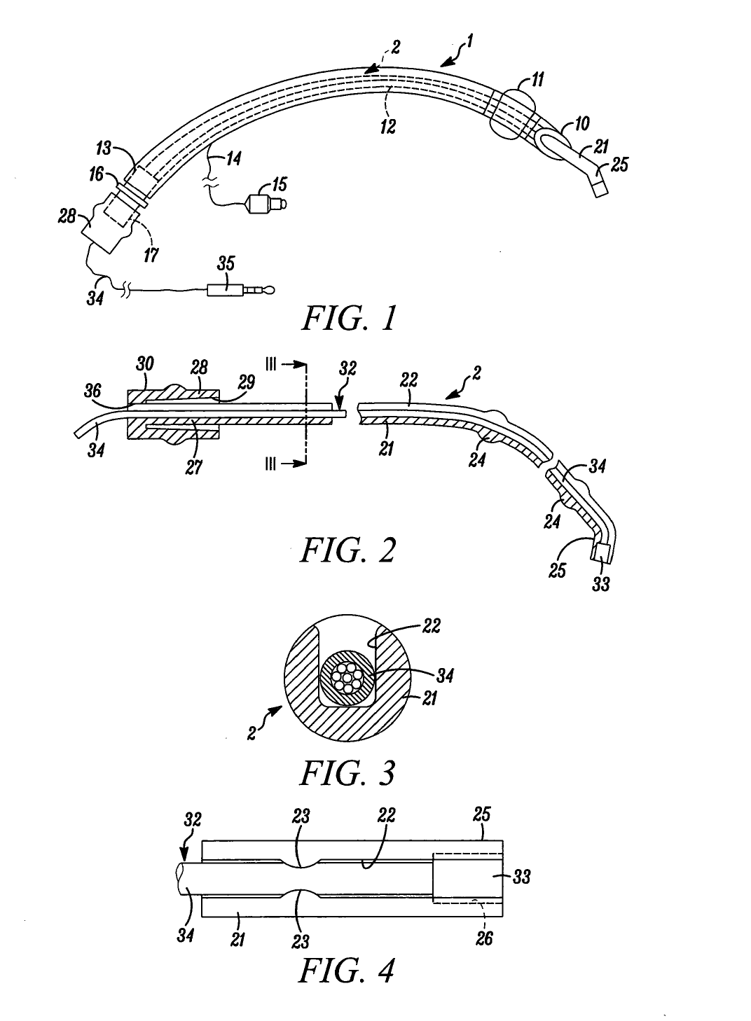

FIG. 1 is a side elevation view of the assembly;

FIG. 2 is an enlarged sectional side elevation view of the video introducer;

FIG. 3 is a cross-sectional transverse view of the introducer along the line of FIG. 2;

FIG. 4 is an enlarged plan view of the forward end of the introducer; and

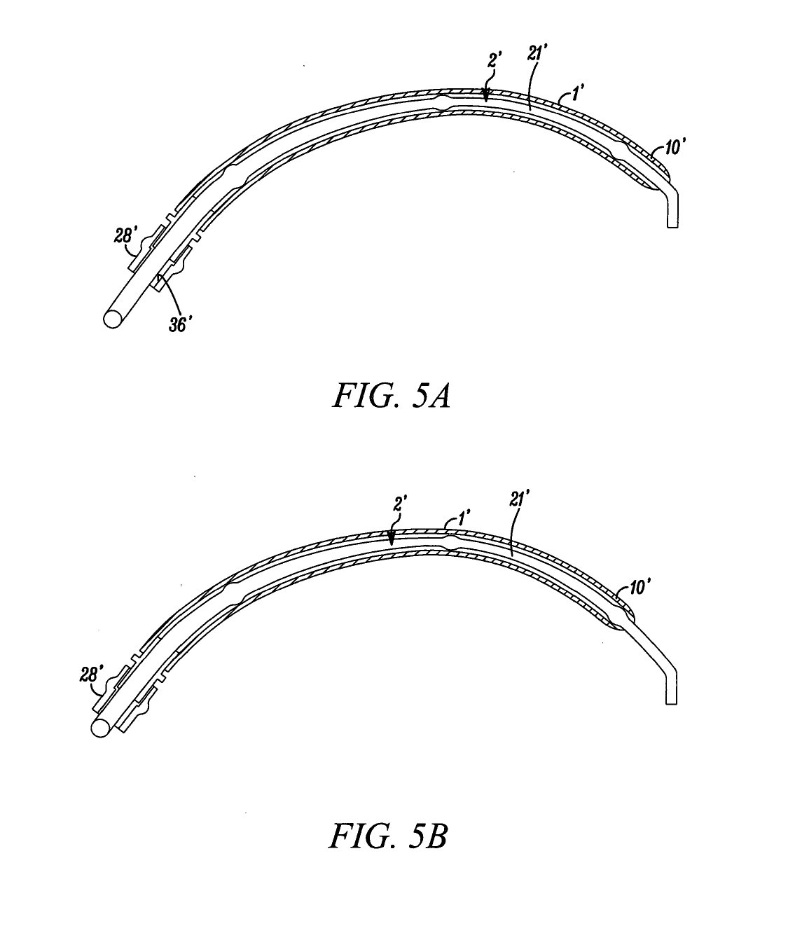

FIGS. 5A and 5B illustrate an alternative introducer in two different states.

With reference first to FIG. 1, the assembly includes a conventional endotracheal tube 1 and a visualisation device or means in the form of a video introducer 2 inserted within the tube.

The endotracheal tube 1 has a bevelled forward, patient end tip 10 and an optional inflatable sealing cuff 11 spaced a short distance rearwardly of the tip. The cuff 11 is inflated and deflated via a small bore inflation lumen 12 extending within the wall of the tube and connected towards the rear, machine end 13 of the tube 1 with an inflation line 14 extending to a combined inflation indicator, valve and connector 15. A conventional breathing circuit connector 16 is fitted in the rear end 13 of the tube 1, the connector having male terminations at opposite ends and with a 15 mm male luer tapered termination 17 at its rear end. As so far described the tube 1 is conventional.

The video introducer 2 is shown most clearly in FIGS. 2 to 4. The introducer 2 includes a shaft 21 of circular section along most of its length and with an external diameter less than the internal diameter of the tube 1. Shafts of alternative sections could be used. The shaft 21 is formed with a channel 22 that opens along its length and that has several short sections of reduced width towards its outer surface spaced along the shaft so as to form retaining clips 23. The width of the shaft 21 is less than the internal diameter of the tube 1 apart from several enlarged bosses 24 moulded with the shaft, such as midway along its length and towards its patient end, so as to centralise the shaft within the bore of the tube 1. At its forward, patient end 25 the shaft 21 has a cavity or recess 26. At its rear, patient end 27 the shaft 21 is joined with a connector 28 having a forwardly opening recess 29 that provides a female luer tapered surface adapted to mate with the male tapered surface 17 on the coupling 16 fitted in the endotracheal tube 1. The external surface 30 of the connector 28 is shaped to provide an ergonomic profile to enable the connector to be gripped in the hand and used to manipulate the assembly. The connector 28 could be overmoulded with a softer plastics material to give it a softer surface to enhance the grip with the connector. The connector 28 and shaft 21 are preferably integrally moulded together from a plastics material as a single piece, such as by injection moulding. The material used is selected to give the shaft 21 the desired properties and, in this respect, may be chosen to be similar to endotracheal tube bougies that can be bent to the desired shape and then relax to a less deformed shape, or it could be of a material that retains a permanent set after being pre-shaped by the user. The shaft 21 could be modified in various ways to give it the desired mechanical properties such as by the incorporation of softer materials or by including a length of a malleable material to enable the shaft to be bent to and retain a desired shape along its length, or along a part of its length.

The video introducer is completed by an imaging assembly in the form a camera assembly 32 comprising an imaging device or means in the form of a camera module 33, an electrical cable 34 and a plug or other form of electrical signal connector 35. The camera module 33 may include any conventional video camera chip and processing, and preferably also includes illumination means in the form of a lamp, such as provided by one or more LEDs. The camera module 33 is fitted in the recess 26 at the end of the shaft 21 and is preferably bonded in place by an adhesive or solvent. The camera module 33 could be enclosed within a separate transparent cap (not shown) attached onto the patient end of the shaft. Instead of using a video camera, the imaging means could be provided by one end of a fibre-optic cable extending along the shaft 21 to an optical connector. The patient end 25 of the shaft 21 may be pre-formed with a Coude bent tip and could be overmoulded with a softer plastics material or otherwise provided with a soft, atraumatic surface. The length of the shaft 21 is chosen so that the camera module 33 is located close enough to the patient end tip 10 of the tube 1 when inserted to ensure that the camera module has a clear view of the region of interest. Preferably, as shown, the shaft 21 extends beyond the tip 10 of the tube 1 by a short distance so that the field of view of the camera 33 is not obstructed by the end of the tube or the sealing cuff 11.

The major part of the length of the cable 34 is loaded into the channel 22 along the shaft 21 from its side simply by pushing it into the opening of the channel. The diameter of the channel 22 is chosen so that the cable 34 is a close fit and so that it can be pushed past the retaining clips 23 along the channel, either by the insulation on the cable deforming or by the channel being deformed outwardly resiliently. An adhesive or solvent may be used to retain the cable 34 permanently in the channel 22. A filler may be applied along the top of the channel 22 after loading the cable 34 so as seal it in the shaft 21 and provide electrical insulation. The cable could be retained in the shaft in other ways, such as by a lid fitted in and bonded to the channel. The plug 35 at the end of the cable 34 is threaded through an enlarged aperture 36 in the end of the connector 28 so that the cable extends loose beyond the introducer 2, enabling the plug to be connected with a mating connector for electrical connection with a display (not shown).

The introducer 2 enables the camera assembly 32 of the camera module 33, cable 34 and plug 35 to be supplied complete, ready assembled and tested, and subsequently fitted into the channel 22 along the shaft 21. This avoids the need to connect the plug or camera module to the cable after assembly in the introducer, when it would be more difficult to achieve reliably.

In use, the assembly of the tube 1 and introducer 2 can be supplied ready assembled in a single sterile pack or the user could insert an introducer into a tube as needed. After the plug 35 on the video introducer 2 is connected to a suitable display the clinician can visually monitor insertion of the assembly into the trachea while the introducer retains the desired shape of the tube 1 and provides a leading extension at the patient end of the assembly for use while inserting the assembly into the trachea. The aperture 36 in the introducer connector 28 can be chosen to be sufficiently large to provide an air passage that allows air to flow through the tube 1 during insertion. The enlarged external surface 30 of the connector 28 on the introducer 2 forms a useful handle by which the user can grip and manipulate the assembly. After insertion, the introducer 2 is simply disconnected from the connector 16 and pulled out of the tube 1.

FIGS. 5A and 5B illustrate a modified assembly where the shaft 21′ of the introducer 2′ is formed separately of the connector 28′ and extends in a slidable manner through an opening 36′ in the connector so that a length of the shaft protrudes rearwardly to provide a handle. When the rear end of the shaft 21′ is pulled rearwardly to its full extent the forward end of the shaft only projects a short distance from the patient end 10′ of the tube 1′, as shown in FIG. 5A. The shaft 21′ can be pushed forwardly, into the tube 1′, through the connector 28′, as shown in FIG. 5B, when the user needs a longer extension of the introducer beyond the patient end 10′ of the tube.

The relative low cost of the video introducer enables it to be disposed of after a single use. The connector 16 on the tube 1 can then be left open after insertion if the patient is breathing normally or connected to a breathing circuit if the patient requires ventilation or anaesthesia. The invention is not confined to endotracheal tubes but could be used with other airway tubes, such as tracheostomy tubes and laryngeal masks.

1-15. (canceled)

16. An assembly including an airway tube and a first connector fitted onto the rear end of the tube, characterized in that the assembly includes a visualization device including an imaging device at one end, a shaft extending along the visualization device, a second connector towards the rear end of the shaft and matable with the first connector, and a cable extending along the shaft from the imaging device to a signal connector located to the rear of the second connector, the second connector being removably fitted with the first connector on the tube and such that the imaging device is located towards the forward, patient end of the tube to view the field of view forwardly of the patient end of the tube.

17. An assembly according to claim 16, characterized in that the imagining device includes a video camera, that the cable is an electrical cable and the signal connector is an electrical connector.

18. An assembly according to claim 16, characterized in that the imaging device is provided by one end of an optical fiber cable, and that the signal connector is an optical connector.

19. An assembly according to claim 16, characterized in that the shaft has a channel opening along its length, and that at least a major part of the length of the cable is fitted in the channel.

20. An assembly according to claim 19, characterized in that the channel has a plurality of clip formations along its length to retain the cable in the channel.

21. An assembly according to claim 16, characterized in that the visualization device projects beyond the forward end of the tube such that the imaging device is located forwardly of the forward end of the tube.

22. An assembly according to claim 21, characterized in that a length of the visualization device projecting beyond the forward end of the tube provides a leading extension for use during insertion of the assembly.

23. An assembly according to claim 16, characterized in that the shaft has a width and the tube has a bore, the width of the shaft being less than the bore of the tube apart from a plurality of enlarged bosses that help centralize the shaft in the bore of the tube.

24. An assembly according to claim 16, characterized in that the second connector includes an opening that allows air to flow through the tube during use.

25. An assembly according to claim 16 , characterized in that the first connector has a male fitting at one end that is fitted in the machine end of the tube and a male fitting at its opposite end that is fitted in the second connector.

26. An assembly according to claim 16, characterized in that the second connector has an opening therethrough, and that the signal connector is threaded through the opening.

27. An assembly according to claim 16, characterized in that the second connector has an external surface that provides a handle by which the assembly can be gripped and manipulated.

28. An assembly according to claim 16, characterized in that the shaft and the second connector are integrally formed with one another as a single piece.

29. An assembly according to claim 16, characterized in that the shaft extends rearwardly beyond the second connector and is slidable relative to the second connector.

30. A visualization device for an assembly comprising an airway tube and a first connector fitted onto the rear end of the tube, wherein the visualization device includes an imaging device at one end, the assembly device further comprising a shaft extending along the visualization device, a second connector towards the rear end of the shaft and matable with the first connector, and a cable extending along the shaft from the imaging device to a signal connector located to the rear of the second connector, the second connector being removably fitted with the first connector on the tube and such that the imaging device is located towards the forward, patient end of the tube to view the field of view forwardly of the patient end of the tube.
